(19) United States
(12) Patent Application Publication
(10) Pub. No.: US 2025/0121144 A1
(43) Pub. Date: Apr. 17, 2025

(54) VORTEX TUBE ASSEMBLY AND BREATHING DEVICE

(71) Applicant: RESVENT MEDICAL TECHNOLOGY CO., LTD, Shenzhen (CN)

(72) Inventors: Kai QU, Shenzhen (CN); Rongrong ZHU, Shenzhen (CN)

(21) Appl. No.: 18/896,889

(22) Filed: Sep. 26, 2024

(30) Foreign Application Priority Data
Oct. 16, 2023 (CN) 202311335792.3

(51) Int. Cl. A61M 16/00; A61M 16/10; A61M 16/20

(52) U.S. Cl. CPC A61M 16/0066; A61M 16/0003; A61M 16/109; A61M 16/203; A61M 2016/0027; A61M 2016/003; A61M 2202/0007; A61M 2202/0208; A61M 2205/103; A61M 2205/3327; A61M 2205/3334; A61M 2205/362; A61M 2206/16

(57) ABSTRACT

A vortex tube assembly and a breathing device include a main tube and a hot end plug. Gas inlet slots are annularly defined on a side surface of the main tube, so that compressed gas entering the main tube forms a vortex airflow. A first end of the main tube is a cold gas outlet connected to a turbine fan to dissipate heat. A second end of the main tube is a hot gas outlet. The hot end plug is mounted on the hot gas outlet of the main tube. A middle portion of the hot end plug blocks the vortex airflow close to an axis of the main tube, and hot gas outlet annular holes are defined around the middle portion of the hot end plug, so that the vortex airflow close to a tube wall of the main tube flows out from the hot gas outlet.

### CROSS-REFERENCE TO RELATED APPLICATION

This application claims priority to Chinese Patent Application No. 202322604204.3, titled “VORTEX TUBE ASSEMBLY AND BREATHING DEVICE” and filed to the China National Intellectual Property Administration on Sep. 25, 2023, the entire contents of which are incorporated herein by reference.

### TECHNICAL FIELD

The present disclosure relates to a technical field of medical devices, and in particular to a vortex tube assembly and a breathing device.

### BACKGROUND

A ventilator is a medical device configured to assist or maintain a patient's breathing. The ventilator assists or replaces the patient's spontaneous breathing by delivering gas (usually a mixture of air and oxygen). The ventilator commonly comprises a gas source, a gas delivery tube, a gas heating and humidification device, a gas flow sensor, and a breathing circuit connected to the patient. There are two solutions for heat dissipation of the turbine fan of a conventional ventilator. A first solution is to dissipate heat by sucking air from environment through the turbine fan itself. A second solution is to transfer the heat of the turbine fan to an outside through a metal radiator, and then the heat is dissipated through forced air cooling of a cooling fan. However, a heat dissipation effect is limited by an amount of the air entering the turbine fan when the turbine fan itself sucks the air from the environment to dissipate heat, and the first solution has limited effect when the turbine fan is under high load or generates a large amount of heat, so the first solution is only applicable to turbine fans with small load or turbine fans generating a small amount of heat. In the second solution of transferring the heat of the turbine fan to the outside through the metal radiator and dissipating the heat through the cooling fan, a heat dissipating system thereof has a complex structure, many parts, and high cost.

### SUMMARY

In order to solve a technical problem of low heat dissipation efficiency or high cost of a turbine fan in the prior art, the present disclosure proposes a vortex tube assembly and a breathing device.

The present disclosure provides the vortex tube assembly. The vortex tube assembly comprises a main tube, a gas inlet ring, and a hot end plug.
gas inlet slots are annularly defined on a side surface of the main tube, so that compressed gas entering the main tube from the gas inlet slots forms a vortex airflow in the main tube. A first end of the main tube is a cold gas outlet. The cold gas outlet is connected to an air inlet of a turbine fan to dissipate heat of the turbine fan. A second end of the main tube is a hot gas outlet.

The gas inlet ring is mounted on an outer side of the main tube and covering the gas inlet slots. a gas inlet connector is defined on the gas inlet ring.

The hot end plug is mounted on the hot gas outlet of the main tube. A middle portion of the hot end plug blocks the vortex airflow close to an axis of the main tube, and hot gas outlet annular holes are defined surround the middle portion of the hot end plug, so that the vortex airflow close to a tube wall of the main tube flows out from the hot gas outlet.

Furthermore, an annular groove is defined on an inner wall of the gas inlet ring. The gas inlet connector is defined on an outer wall of the gas inlet ring and penetrates to the annular groove. When the gas inlet ring is sleeved on the main tube, a gas inlet cavity communicating with the gas inlet slots is formed between the annular groove and an outer wall of the main tube.

Furthermore, the gas inlet slots are defined close to the cold gas outlet.

Furthermore, an aperture of the cold gas outlet is less than an aperture of the hot gas outlet. The cold gas outlet directly faces the middle portion, blocking the vortex airflow, of the hot end plug.

Furthermore, the hot end plug comprises a connecting portion and a plug, the connecting portion is connected to the main tube. The plug is disposed at a middle portion of one end of the connecting portion directly facing the vortex airflow. The hot gas outlet annular holes surround the middle portion of the one end of the connecting portion directly facing the vortex airflow.

Furthermore, the hot gas outlet of the main tube is trumpet-shaped. The plug is inserted into the main tube. The plug is in a circular truncated cone shape. The hot gas outlet annular holes are formed between the plug and a tube wall of the main tube.

The present disclosure further provides the breathing device. The breathing device comprises the vortex tube assembly mentioned above.

In a first embodiment, the breathing device further comprises the turbine fan, a humidifier, a heating tube, a flow sensor, and a pressure sensor.

An air inlet of the turbine fan is communicated with an air inlet of the breathing device. An air outlet of the turbine fan is communicated with the gas inlet connector of the gas inlet ring. A front end of the humidifier is communicated with the air outlet of the turbine fan. A rear end of the humidifier is connected to a pipeline connected to a user. An inlet end of the heating tube is connected to the hot gas outlet of the main tube. The heating tube is disposed on a bottom potion of the humidifier to heat the humidifier. An outlet end of the heating tube is communicated with a front end of the humidifier. The flow sensor is configured to measure a flow rate of airflow entering the breathing device. The pressure sensor is configured to measure an air pressure of the air outlet of the turbine fan.

In a second embodiment, the breathing device further comprises a turbine fan, a humidifier, a heating tube, a high-pressure oxygen branch, a flow sensor, and a pressure sensor.

An air inlet of the turbine fan is communicated with an air inlet of the breathing device. A front end of the humidifier is communicated with an air outlet of the turbine fan. A rear end of the humidifier is connected to a pipeline connected to a user. An inlet end of the heating tube is connected to the hot gas outlet of the main tube. The heating tube is disposed on a bottom potion of the humidifier to heat the humidifier. An outlet end of the heating tube is communicated with the air inlet of the turbine fan. The high-pressure oxygen branch is connected to the gas inlet connector of the gas inlet ring. A proportional valve and an oxygen flow sensor are disposed in the high-pressure oxygen branch. The flow sensor is configured to measure a flow rate of airflow entering or flowing out of the turbine fan. The pressure sensor is configured to measure an air pressure of the air outlet of the turbine fan.

Optionally, the breathing device is a ventilator or a high-flow oxygen therapy device.

Compared with the prior art, in the present disclosure, the vortex tube assembly is driven by compressed gas and separates the compressed gas into cold gas and hot gas, and the cold gas and the hot gas are output from the cold gas outlet and the hot gas outlet respectively. The cold gas from the cold gas outlet is input to the air inlet of the turbine fan to dissipate the heat of the turbine fan, and the hot gas from the hot gas outlet is input to the heating tube to heat the humidifier.

The vortex tube assembly realizes heat dissipation of the turbine fan and heating of the humidifier while separating the compressed gas. Compared with the prior art, the vortex tube assembly realize the separation of the cold gas and the hot gas only by the compressed gas and a structure of the vortex tube assembly itself, rather than a complex structure, making the structure of the vortex tube assembly simpler and cost of the vortex tube assembly lower.

### BRIEF DESCRIPTION OF DRAWINGS

In order to clearly describe technical solutions in the embodiments of the present disclosure, the following will briefly introduce the drawings that need to be used in the description of the embodiments or the prior art. Apparently, the drawings in the following description are merely some of the embodiments of the present disclosure, and those skilled in the art are able to obtain other drawings according to the drawings without contributing any inventive labor.

FIG. 1 is a perspective schematic diagram of a vortex tube assembly according to one embodiment of the present disclosure.

FIG. 2 is an exploded schematic diagram of the vortex tube assembly according to one embodiment of the present disclosure.

FIG. 3 is a side cross-sectional schematic diagram of the vortex tube assembly according to one embodiment of the present disclosure.

FIG. 4 is a cross-sectional schematic diagram taken from a gas inlet ring of the vortex tube assembly according to one embodiment of the present disclosure.

FIG. 5 is a schematic diagram showing flowing path of vortex airflow in the vortex tube assembly according to one embodiment of the present disclosure.

FIG. 6 is a schematic diagram showing flowing path of airflow at the gas inlet ring of the vortex tube assembly according to one embodiment of the present disclosure.

FIG. 7 is a perspective schematic diagram of a hot end plug according to one embodiment of the present disclosure.

FIG. 8 is a cross-sectional schematic diagram of the hot end plug according to one embodiment of the present disclosure.

FIG. 9 is a front side schematic diagram of the hot end plug according to one embodiment of the present disclosure.

FIG. 10 is a perspective schematic diagram of a main tube according to one embodiment of the present disclosure.

FIG. 11 is a cross-sectional schematic diagram of the main tube according to one embodiment of the present disclosure.

FIG. 12 is a block diagram of components of a breathing device according to a first embodiment of the present disclosure.

FIG. 13 is a block diagram of components of the breathing device according to a second embodiment of the present disclosure.

FIG. 14 is another block diagram of components of the breathing device according to the second embodiment of the present disclosure.

In the drawings:

100-vortex tube assembly; 1-main tube; 11-cold gas outlet; 12-hot gas outlet; 13-gas inlet slot; 2-gas inlet ring; 21-gas inlet connector; 22-annular groove; 3-hot end plug; 31-plug; 32-connecting portion; 33-hot gas outlet annular hole.

### DETAILED DESCRIPTION

In order to make technical problems, technical solutions, and beneficial effects to be solved by the present disclosure clearer, the present disclosure is further described in detail below in conjunction with accompanying drawings and embodiments. It should be understood that the specific embodiments described herein are only used to explain the present disclosure and are not used to limit the present disclosure.

The principle and structure of the present disclosure are described in detail below in conjunction with the accompanying drawings and embodiments.

There are two solutions for heat dissipation of a turbine fan of a conventional ventilator. In a first solution, a heat dissipation effect is limited by an amount of the air entering the turbine fan, and the first solution has limited effect when the turbine fan is under high load or generates a large amount of heat, so the first solution is only applicable to turbine fans with small load or turbine fans generating a small amount of heat. In a second solution of transferring the heat of the turbine fan to an outside through a metal radiator and dissipating the heat through a cooling fan, a heat dissipating system thereof has a complex structure, many parts, and high cost. Therefore, the present disclosure proposes a vortex tube assembly for a ventilator or a high-flow humidifier. The vortex tube assembly is driven by compressed gas and separates the compressed gas into cold gas and hot gas, and the cold gas and the hot gas are output from a cold gas outlet and a hot gas outlet respectively. The cold gas from the cold gas outlet is input to the air inlet of a turbine fan to dissipate the heat of the turbine fan, and the hot gas from the hot gas outlet is input to a heating tube to heat a humidifier.

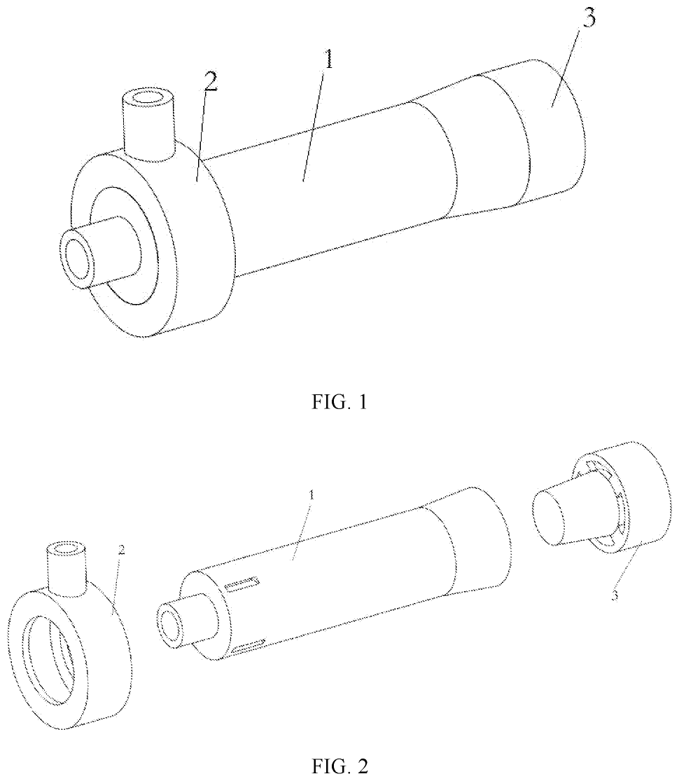

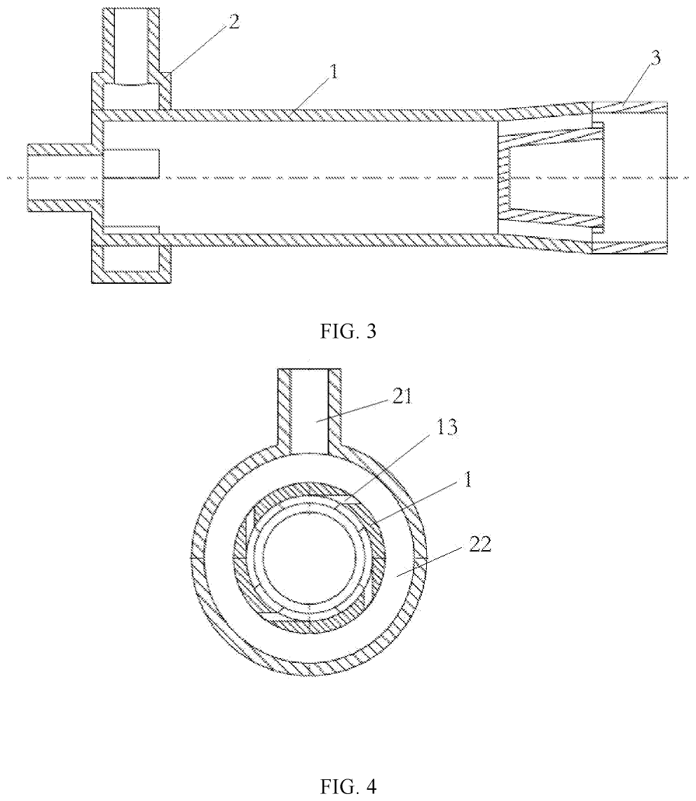

As shown in FIGS. 1-5, the vortex tube assembly 100 of the present disclosure comprises a main tube, a gas inlet ring 2, and a hot end plug 3.

Gas inlet slots 13 are annularly defined on a side surface of the main tube 1. The gas inlet slots 13 are communicated with an interior of the main tube 1, and an angle between a tangent of each of the gas inlet slots 13 and a tube wall of the main tube 1 is less than 90 degrees, so that the compressed gas forms a vortex airflow that is rotating after entering the main tube 1. The vortex airflow close to the tube wall rotates relatively slowly, and the vortex airflow close to a center of the main tube rotates relatively slowly fast. The vortex airflow close to the tube wall of the main tube is high in temperature, and the vortex airflow close to the center of the main tube is low in temperature.

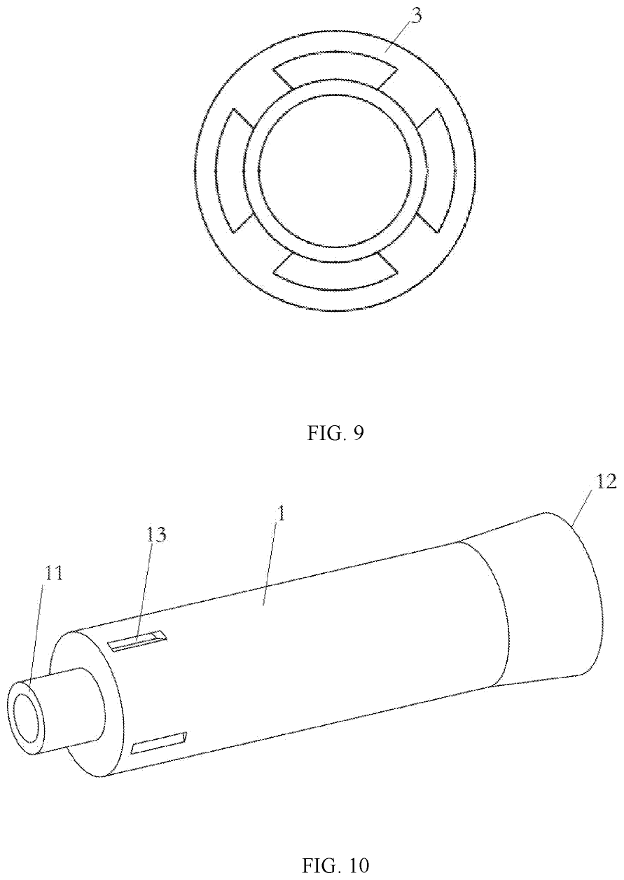

As shown in FIG. 10, a first end of the main tube 1 is a cold gas outlet 11. The cold gas outlet 11 is connected to an air inlet of a turbine fan to dissipate heat of the turbine fan. A second end of the main tube 1 is a hot gas outlet 12. The cold gas outlet 11 is configured to discharge the vortex airflow close to the center of the main tube, and the hot gas outlet 12 is configured to discharge the vortex airflow close to the tube wall of the main tube 1.

The gas inlet ring 2 is sleeved on an outer side of the main tube 1 and covering the gas inlet slots 13. The gas inlet ring 2 and the main tube 1 are sealed and connected. A gas inlet connector 21 is defined on the gas inlet ring 2 and is configured to connect to an external air source. The gas inlet connector 21 is communicated with the gas inlet slots 13.

The hot end plug 3 is mounted on the hot gas outlet 12 of the main tube 1. A middle portion of the hot end plug 3 blocks the vortex airflow close to an axis of the main tube 1, and hot gas outlet annular holes 33 are defined surround the middle portion of the hot end plug 3, so that the vortex airflow close to the tube wall of the main tube 1 flows out. An aperture of the cold gas outlet 11 of the main tube 1 is less than an aperture of the hot gas outlet 12. The cold gas outlet 11 directly faces the middle portion, blocking the vortex airflow, of the hot end plug 3, so that the vortex airflow close to the center of the main tube 1 flows out.

By such arrangements, cold gas and hot gas are separated. The cold gas outlet 11 of the main tube 1 is connected to an air inlet of the turbine fan to dissipate the heat of the turbine fan, and the hot gas outlet 12 is connected to the heating tube to heat the humidifier, so that the vortex tube assembly 100 realizes heat dissipation of the turbine fan and heating of the humidifier while separating the compressed gas through separation of the compressed gas. Compared with the prior art, the vortex tube assembly 100 realize the separation of the cold gas and the hot gas only by the compressed gas and a structure of the vortex tube assembly 100 itself, rather than a complex structure, making the structure of the vortex tube assembly 100 simpler and cost of the vortex tube assembly lower.

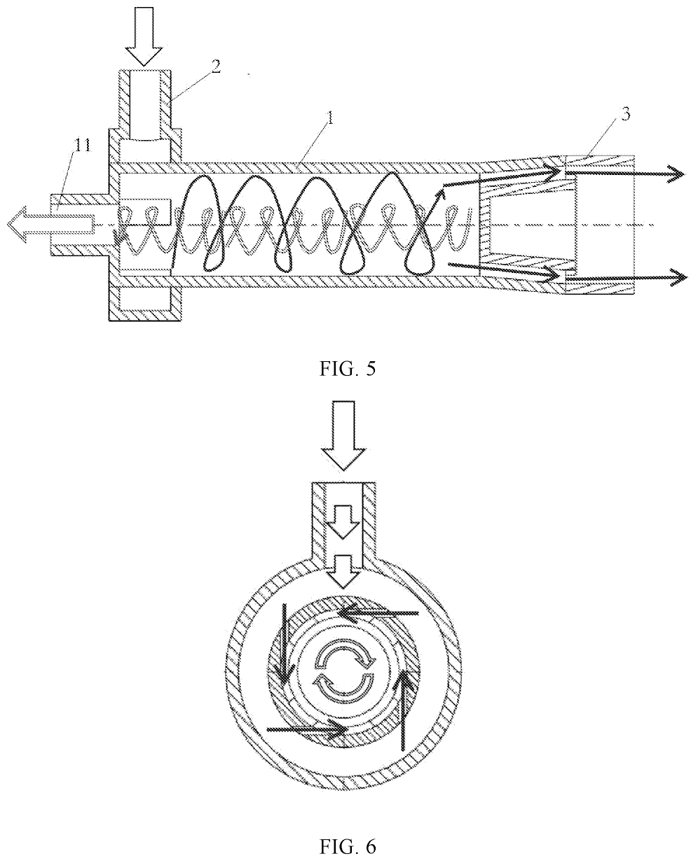

As shown in FIGS. 4 and 5, in one optional embodiment, an annular groove 22 is defined on an inner wall of the gas inlet ring 2. The gas inlet connector 21 is defined on an outer wall of the gas inlet ring 2 and penetrates to the annular groove 22. When the gas inlet ring 2 is sleeved on the main tube 1, the annular groove 22 directly faces the gas inlet slots 13 on the main tube 1, and a gas inlet cavity communicating with the gas inlet slots 13 is formed between the annular groove 22 and an outer wall of the main tube 1.

The gas inlet cavity is annular. The gas inlet cavity is communicated with the gas inlet connector 21 and the gas inlet slots 13. The external air source flows into the gas inlet cavity from the gas inlet connector 21, and then enters the gas inlet slots 13 through the gas inlet cavity to blow air into the main tube 1 along a tangential direction of the gas inlet slots 13 to form the vortex airflow.

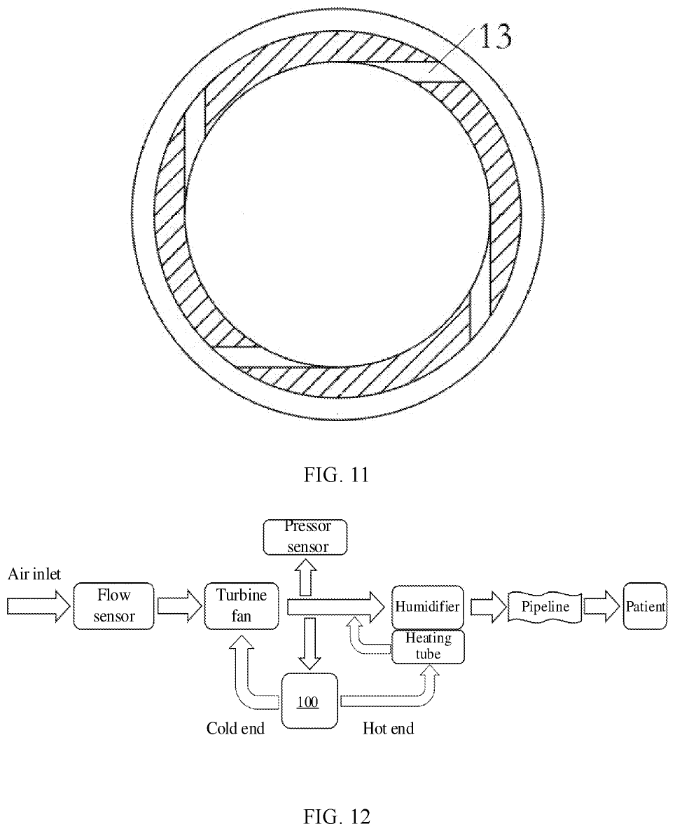

As shown in FIG. 4 and FIG. 11, in one optional embodiment, four gas inlet slots 13 are evenly distributed.

Optionally, the gas inlet slots 13 are defined close to the cold gas outlet 11 of the main tube 1, so that the vortex airflow rotating faster is able to quickly flow out from the cold gas outlet 11 to realize a good separation effect of the cold gas and the hot gas.

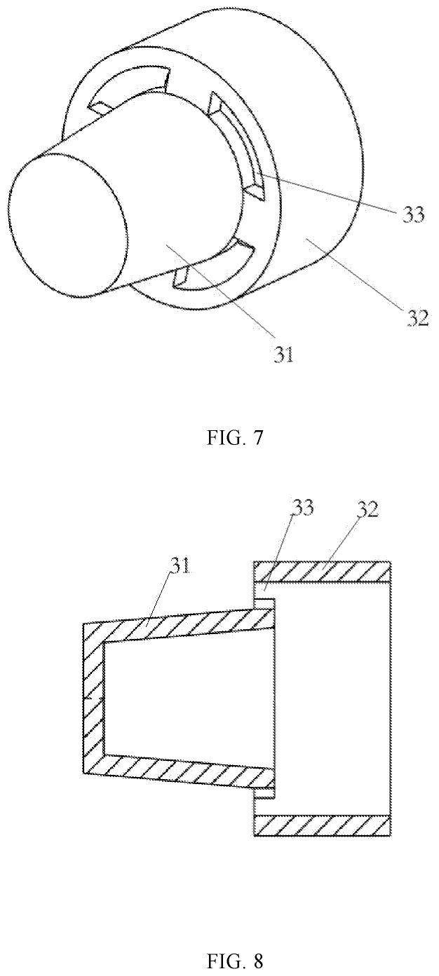

As shown in FIGS. 7-9, in one optional embodiment, the hot end plug 3 comprises a connecting portion 32 and a plug 31. The connecting portion 32 is of a tube shape and is connected to the hot gas outlet of the main tube 1. The plug 31 is disposed at a middle portion of one end of the connecting portion 32 directly facing the main tube 1. The hot gas outlet annular holes 33 surround the middle portion of the one end of the connecting portion 32 directly facing an end surface of the main tube 1. The plug 31 is configured to block the vortex airflow close to the axis of the main tube 1, and the hot gas outlet annular holes 33 are configured to discharge the vortex airflow close to the tube wall of the main tube 1.

In one optional embodiment, four hot gas outlet annular holes 33 are evenly distributed.

Furthermore, the hot gas outlet 12 of the main tube 1 is trumpet-shaped. The connecting portion 32 is connected to the hot gas outlet 12 of the main tube 1. The plug 31 is inserted into the main tube 1. The plug 31 is in a circular truncated cone shape. A hot gas outlet 12 gap is formed between the plug and a tube wall of the main tube 1, which further improves separation efficiency of the hot gas and the cold gas.

The present disclosure further provides the breathing device. The breathing device comprises the vortex tube assembly 100 mentioned above.

Optionally, the breathing device is a ventilator, a high-flow oxygen therapy device, or a high-flow humidification device

As shown in FIG. 12, in a first embodiment, the breathing device comprises the vortex tube assembly 100, the turbine fan, a humidifier, a heating tube, a flow sensor, and a pressure sensor.

An air inlet of the turbine fan is communicated with an air inlet of the breathing device. An air outlet of the turbine fan is communicated with the gas inlet connector 21 of the gas inlet ring 2 of the vortex tube assembly 100. A front end of the humidifier is communicated with the air outlet of the turbine fan. A rear end of the humidifier is connected to a pipeline connected to a patient. An inlet end of the heating tube is connected to the hot gas outlet 12 of the main tube 1. The heating tube obtains the hot gas separated by the main tube 1. The heating tube is disposed on a bottom potion of the humidifier to heat water in the humidifier. An outlet end of the heating tube is communicated with a front end of the humidifier. That is, the hot gas from the heating tube continues to be used by the humidifier after the heat thereof is dissipated. The flow sensor is configured to measure a flow rate of airflow entering the breathing device. The flow sensor is disposed between the air inlet of the turbine fan and the air inlet of the breathing device. The pressure sensor is configured to measure an air pressure of the air outlet of the turbine fan. The pressure sensor is disposed at the air outlet of the turbine fan.

As shown in FIGS. 13-14, in a second embodiment, the breathing device comprises the vortex tube assembly 100, the turbine fan, a humidifier, a heating tube, a high-pressure oxygen branch, a flow sensor, and a pressure sensor.

An air inlet of the turbine fan is communicated with an air inlet of the breathing device. A front end of the humidifier is communicated with an air outlet of the turbine fan. A rear end of the humidifier is connected to a pipeline connected to a user. An inlet end of the heating tube is connected to the hot gas outlet 12 of the main tube 1. The heating tube obtains the hot gas separated by the main tube 1. The heating tube is disposed on a bottom potion of the humidifier to heat water in the humidifier. An outlet end of the heating tube is communicated with the air inlet of the turbine fan. A first end of the high-pressure oxygen branch is connected in high-pressure oxygen inlet, and a second end of the high-pressure oxygen branch is connected to the gas inlet connector 21 of the gas inlet ring 2. The second end of high-pressure oxygen branch is served as an air source. A proportional valve and an oxygen flow sensor are disposed in the high-pressure oxygen branch. The proportional valve and the oxygen flow sensor realize control and detection of a flow rate of the high-pressure oxygen.

The flow sensor is configured to measure a flow rate of airflow entering or flowing out of the turbine fan. The flow sensor is disposed between the air inlet of the turbine fan and the air inlet of the breathing device. Alternatively, the flow sensor is disposed at the air outlet of the turbine fan The pressure sensor is configured to measure an air pressure of the air outlet of the turbine fan. The pressure sensor is disposed at the air outlet of the turbine fan.

It should be noted that the terms used in the present disclosure are for a purpose of describing particular embodiments only and does not limit the present disclosure. As used in the present disclosure, singular forms are intended to comprise the plural forms as well, unless the context clearly dictates otherwise. It is also understood that the term “and/or” as used herein refers to and comprises features, steps, operations, devices, components and/or combinations thereof.

Unless specifically stated otherwise, relative arrangements of components, steps, numerical expressions, and numerical values set forth in these embodiments do not limit the scope of the present disclosure. In addition, it should be understood that, for case of description, the sizes of the parts shown in the accompanying drawings are not drawn according to actual proportional relationships. Techniques, methods, and apparatus known to those skilled in the art may not be discussed in detail, but where appropriate, the techniques, methods, and apparatus should be considered as part of the authorization specification. In all embodiments shown and discussed herein, any specific value should be construed as exemplary only and not as a limitation. Thus, other examples of the exemplary embodiments may have different values. It should be noted that similar reference numerals and letters refer to similar items in the following drawings, and therefore, once an item is defined in one figure, it does not need to be further discussed in the subsequent figures.

It should be understood in the description of the present disclosure that directional terms such as “front”, “rear”, “upper”, “lower”, “left”, “right”, “vertical”, “horizontal”, “perpendicular|”, “top”, “bottom”, etc. indicate direction or position relationships shown based on the drawings, and are only intended to facilitate the description of the present disclosure and the simplification of the description rather than to indicate or imply that the indicated device or element must have a specific direction or constructed and operated in a specific direction, and therefore, shall not be understood as a limitation to the present disclosure. The directional terms “inside” and “outside” refer to the inside and outside relative to the outline of each component itself.

For ease of description, spatially relative terms, such as “above”, “on”, “on a surface”, “over”, etc., may be used to describe spatial positional relationships between one device or feature and other devices or features as shown in the figures. It should be understood that the spatially relative terms are intended to encompass different orientations in use or operation in addition to the orientation depicted in the figures. For example, if a device in the accompanying drawings is inverted, another device described as “above” or “on” the device is positioned as under the device or below the device. Thus, the exemplary term “above the device” may comprise two orientations such as above the device or below the device. The device may also be positioned (rotated 90 degrees or at other orientations) in other different ways, and the spatially relative description used herein is interpreted accordingly.

In addition, it should be noted that the terms such as “first” and “second” are used to limit the parts, and are merely to distinguish corresponding parts. Unless otherwise stated, the above terms have no special meaning and therefore cannot be understood as limiting the protection scope of the present disclosure.

The above description is only optional embodiments of the present disclosure and is not intended to limit the present disclosure. For those skilled in the art, the present disclosure may have various modifications and variations. Any modification, equivalent replacement, improvement, etc. made within the spirit and principle of the present disclosure shall be comprised in the protection scope of the present disclosure.

What is claimed is:

1. A vortex tube assembly, comprising:
a main tube,
a gas inlet ring, and
a hot end plug;
wherein gas inlet slots are annularly defined on a side surface of the main tube, so that compressed gas entering the main tube from the gas inlet slots forms a vortex airflow in the main tube; a first end of the main tube is a cold gas outlet; the cold gas outlet is connected to an air inlet of a turbine fan to dissipate heat of the turbine fan; a second end of the main tube is a hot gas outlet;
wherein the gas inlet ring is mounted on an outer side of the main tube and covering the gas inlet slots; a gas inlet connector is defined on the gas inlet ring;
wherein the hot end plug is mounted on the hot gas outlet of the main tube; a middle portion of the hot end plug blocks the vortex airflow close to an axis of the main tube, and hot gas outlet annular holes are defined around the middle portion of the hot end plug, so that the vortex airflow close to a tube wall of the main tube flows out from the hot gas outlet.

2. The vortex tube assembly according to claim 1, wherein an annular groove is defined on an inner wall of the gas inlet ring; the gas inlet connector is disposed on an outer wall of the gas inlet ring and penetrates to the annular groove; when the gas inlet ring is sleeved on the main tube, a gas inlet cavity communicating with the gas inlet slots is formed between the annular groove and an outer wall of the main tube.

3. The vortex tube assembly according to claim 1, wherein the gas inlet slots are defined close to the cold gas outlet.

4. The vortex tube assembly according to claim 1, wherein an aperture of the cold gas outlet is less than an aperture of the hot gas outlet; the cold gas outlet directly faces the middle portion, blocking the vortex airflow, of the hot end plug.

5. The vortex tube assembly according to claim 1, wherein the hot end plug comprises a connecting portion and a plug; the connecting portion is connected to the main tube; the plug is disposed at a middle portion of one end of the connecting portion directly facing the vortex airflow; the hot gas outlet annular holes surround the middle portion of the one end of the connecting portion directly facing the vortex airflow.

6. The vortex tube assembly according to claim 5, wherein the hot gas outlet of the main tube is trumpet-shaped; the plug is inserted into the main tube; the plug is in a circular truncated cone shape; the hot gas outlet annular holes are formed between the plug and a tube wall of the main tube.

7. A breathing device, comprising: the vortex tube assembly according to claim 1.

8. The breathing device according to claim 7, wherein the breathing device further comprises the turbine fan, a humidifier, a heating tube, a flow sensor, and a pressure sensor,
wherein an air inlet of the turbine fan is communicated with an air inlet of the breathing device; an air outlet of the turbine fan is communicated with the gas inlet connector of the gas inlet ring; a front end of the humidifier is communicated with the air outlet of the turbine fan; a rear end of the humidifier is connected to a pipeline connected to a user; an inlet end of the heating tube is connected to the hot gas outlet of the main tube; the heating tube is disposed on a bottom potion of the humidifier to heat the humidifier; and an outlet end of the heating tube is communicated with a front end of the humidifier; the flow sensor is configured to measure a flow rate of airflow entering the breathing device; the pressure sensor is configured to measure an air pressure of the air outlet of the turbine fan.

9. The breathing device according to claim 7, wherein the breathing device further comprises the turbine fan, a humidifier, a heating tube, a high-pressure oxygen branch, a flow sensor, and a pressure sensor;
wherein an air inlet of the turbine fan is communicated with an air inlet of the breathing device; a front end of the humidifier is communicated with an air outlet of the turbine fan; a rear end of the humidifier is connected to a pipeline connected to a user; an inlet end of the heating tube is connected to the hot gas outlet of the main tube; the heating tube is disposed on a bottom potion of the humidifier to heat the humidifier; and an outlet end of the heating tube is communicated with the air inlet of the turbine fan; the high-pressure oxygen branch is connected to the gas inlet connector of the gas inlet ring, a proportional valve and an oxygen flow sensor are disposed in the high-pressure oxygen branch; the flow sensor is configured to measure a flow rate of airflow entering or flowing out of the turbine fan; the pressure sensor is configured to measure an air pressure of the air outlet of the turbine fan.

10. The breathing device according to claim 7. wherein the breathing device is a ventilator or a high-flow oxygen therapy device.
